(19) United States
(12) Patent Application Publication
(10) Pub. No.: US 2024/0165366 A1
(43) Pub. Date: May 23, 2024

(54) HUMIDIFYING WATER TANK AND VENTILATION THERAPY EQUIPMENT

(71) Applicant: RESVENT MEDICAL TECHNOLOGY CO., LTD, Shenzhen (CN)

(72) Inventors: Jiefeng Cao, Shenzhen (CN); Rongrong Zhu, Shenzhen (CN)

(73) Assignee: RESVENT MEDICAL TECHNOLOGY CO., LTD, Shenzhen (CN)

(21) Appl. No.: 18/230,824

(22) Filed: Aug. 7, 2023

(30) Foreign Application Priority Data
Nov. 23, 2022 (CN) 202211476434.X

(51) Int. Cl. A61M 16/16; F24F 6/16

(52) U.S. Cl. CPC A61M 16/16; F24F 6/16

(57) ABSTRACT

There is a humidifying water tank provided with a shell, a base plate, and a fan blade. The shell is provided with an opening, an air outlet, and an air inlet. The base plate covers the opening and encloses a receiving cavity together with the shell. The receiving cavity is in communication with the air outlet and the air inlet respectively. The receiving cavity is configured to receive liquid, the base plate is heated by an external heating component to enable the liquid being heated to form vapor. The fan blade is accommodated and rotates in the receiving cavity. Airflow enters the receiving cavity from the air inlet and is mixed with the vapor to form mixed gas, the mixed gas being enabled to flow out from the air outlet under the rotation of the fan blade.

### CROSS REFERENCE TO RELATED APPLICATION

This non-provisional patent application claims priority under 35 U. S. C. § 119 from Chinese Patent Application No. 202211476434.x filed on Nov. 23, 2022, the entire content of which is incorporated herein by reference.

### TECHNICAL FIELD

The disclosure relates to medical equipment technologies, in particular to a humidifying water tank and ventilation therapy equipment.

### BACKGROUND

An air flow path in a humidifying water tank of prior art is mainly extended through the design of internal airflow diversion, so as to improve a gas humidification effect. The humidification method for extending the air flow path is passive, that is, water vapor and air are naturally mixed and humidified. When the flow velocity of air is high and the airflow passes through a water pipe for a short time, the humidification rate is low and the humidification effect required by the equipment cannot be achieved. The extension of the air flow path has a significant limitation on the structure of the water tank, and also leads to a low humidification rate.

### SUMMARY

There is a really need to provide a humidifying water tank and ventilation therapy equipment with a higher humidification.

In a first aspect, a humidifying water tank is provided with a shell, abase plate, and a fan blade. One end of the shell is provided with an opening, and the other end of the shell is provided with an air outlet, and an air inlet. The base plate, covers the opening and encloses a receiving cavity together with the shell, the receiving cavity is in communication with the air outlet and the air inlet respectively. The receiving cavity is configured to receive liquid, the base plate is heated by an external heating component to enable the liquid being heated to form vapor. The fan blade is accommodated in the receiving cavity and rotatably mounted on the shell. When airflow enters the receiving cavity from the air inlet, the airflow can push the fan blade to rotate about the fixed column to form wind force, and the airflow and vapor are accelerated and mixed under the rotation of the fan blade to form a mixed gas; the mixed gas can flow out from the air outlet under the action of the wind force generated by the fan blade.

In a second aspect, a ventilation therapy equipment is provided with a housing, and a humidifying water tank accommodated in the housing. The humidifying water tank includes a shell, a base plate, and a fan blade. One end of the shell is provided with an opening, and the other end of the shell is provided with an air outlet, and an air inlet. The base plate, covers the opening and encloses a receiving cavity together with the shell, the receiving cavity is in communication with the air outlet and the air inlet respectively. The receiving cavity is configured to receive liquid, the base plate is heated by an external heating component to enable the liquid being heated to form vapor. The fan blade is accommodated in the receiving cavity and rotatably mounted on the shell. When airflow enters the receiving cavity from the air inlet, the airflow can push the fan blade to rotate about the fixed column to form wind force, and the airflow and vapor are accelerated and mixed under the rotation of the fan blade to form a mixed gas; the mixed gas can flow out from the air outlet under the action of the wind force generated by the fan blade.

In a third aspect, a humidifying water tank is provided with a shell, abase plate, and a fan blade. The shell is provided with an opening, an air outlet, and an air inlet. The base plate covers the opening and encloses a receiving cavity together with the shell. The receiving cavity is in communication with the air outlet and the air inlet respectively. The receiving cavity is configured to receive liquid, the base plate is heated by an external heating component to enable the liquid being heated to form vapor. The fan blade is accommodated in the receiving cavity and rotatably mounted on the shell, Airflow enters the receiving cavity from the air inlet and is mixed with the vapor to form mixed gas, the mixed gas being enabled to flow out from the air outlet under the action of the wind force generated by the fan blade.

In the foregoing embodiment, through the cooperation of the air outlet, the air inlet, and the fan blade, the airflow flowing into the receiving cavity from the air inlet can drive the fan blade in the receiving cavity to rotate through the kinetic energy of the air, and the rotating fan blade can drive the airflow to the air outlet to flow out of the receiving cavity. The base plate can heat the liquid to generate vapor. The rotation of the fan blade can agitate the airflow and vapor in the receiving cavity, so that the air and the vapor are fully mixed to form a mixed gas with a high humidification rate. The mixed gas flows out from the air outlet under the rotation of the fan blade. The process of agitating the airflow and the vapor by the fan blade is beneficial to improving the humidification rate of the humidifying water tank.

### BRIEF DESCRIPTION OF THE DRAWINGS

In order to illustrate the technical solution in the embodiments of the disclosure or the prior art more clearly, a brief description of drawings required in the embodiments or the prior art is given below. Obviously, the drawings described below are only some of the embodiments of the disclosure. For ordinary technicians in this field, other drawings can be obtained according to the structures shown in these drawings without any creative effort.

FIG. 1 illustrates an isometric diagram of of a humidifying water tank in accordance with an embodiment.

FIG. 2 illustrates a section diagram of a humidifying water tank in accordance with an embodiment.

FIG. 3 illustrates a cross-sectional diagram of a humidifying water tank in accordance with an embodiment.

FIG. 4 illustrates an exploded schematic diagram of a humidifying water tank in accordance with an embodiment.

FIG. 5 illustrates an exploded schematic diagram of ventilation therapy equipment in accordance with an embodiment.

### DETAILED DESCRIPTION OF THE EMBODIMENTS

In the description of the present application, it should be understood that the orientations or positional relationships indicated by the terms “length”, “width”, “upper”, “lower”, “front”, “back”, “left”, “right”, “vertical”, “horizontal”, “top”, “bottom”, “inner”, “outer”, and the like are based on the orientations or positional relationships shown in the accompanying drawings, which are merely intended to facilitate and simplify the description of the present application only, but not to indicate or imply that the device or element referred to must have a particular orientation or be constructed and operated in a particular orientation, and therefore, cannot be interpreted as limiting the present application.

In addition, the terms “first” and “second” are merely for the sake of description, and cannot be understood as indicating or implying the relative importance or implicitly indicating the quantity of the indicated technical features. Therefore, the features defined by the terms “first” and “second” may explicitly or implicitly include one or more of these features. In the description of the present application, the term “plurality” refers to two or more, unless otherwise specifically defined.

In the description of the present application, unless otherwise explicitly specified and defined, the terms “mounted”, “coupled”, “connected”, “fixed”, and the like should be construed broadly, for example, the term “connected” may be fixedly connected, detachably connected, or integrated connected; mechanically or electrically connected; directly or indirectly connected through a medium, intercommunication between two elements, or interaction between two elements. Those of ordinary skill in the art may understand the specific meanings of the above terms in the present application according to specific circumstances.

In order to understand the content of the present application more clearly and accurately, detailed descriptions will be provided with reference to the accompanying drawings. The drawings in the specification show examples of embodiments of the present application, where the same reference numerals represent the same elements. It should be understood that the proportions shown in the drawings of the specification are not actual proportions implemented in the present application, which are merely for illustrative purposes, and are not drawn according to original dimensions.

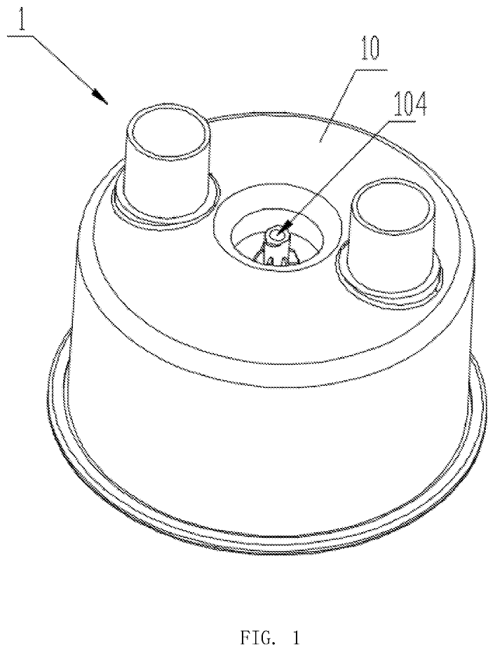

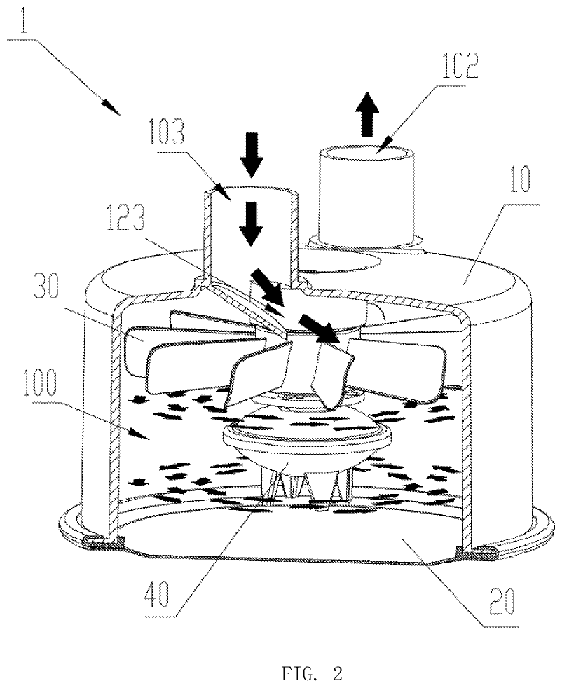

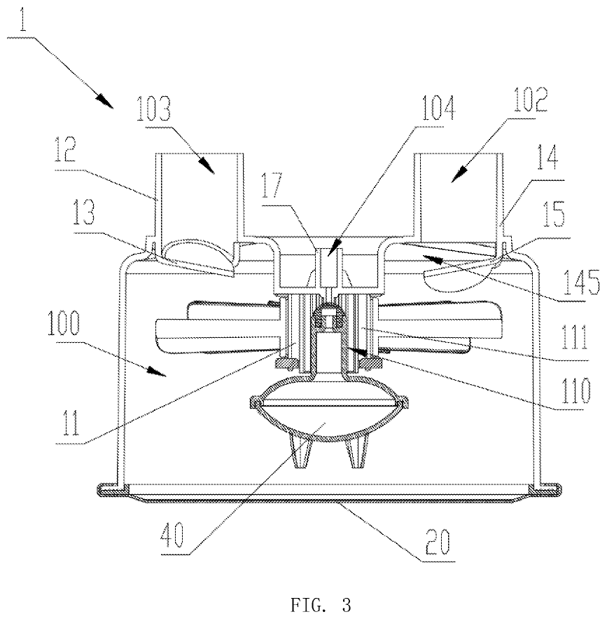

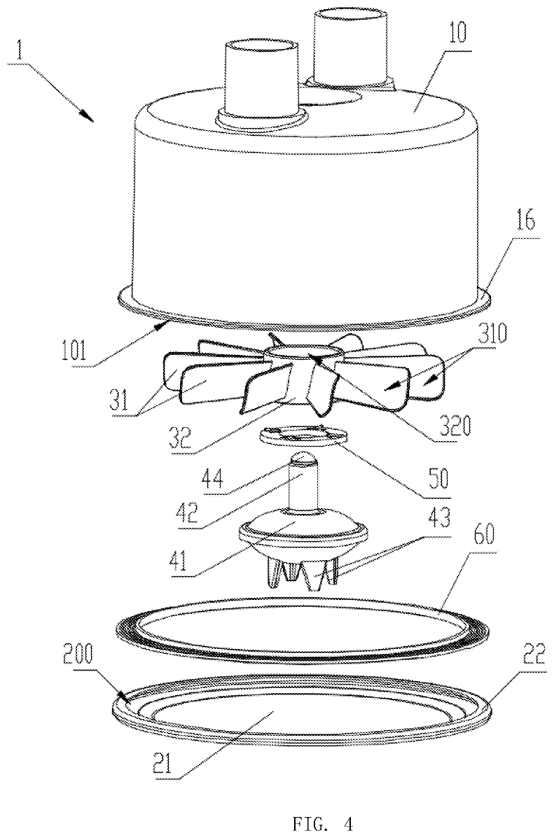

Referring to FIG. 1 to FIG. 4, FIG. 1 is a three-dimensional diagram of a humidifying water tank provided in an embodiment of the present application, FIG. 2 is a section diagram of a humidifying water tank provided in an embodiment of the present application, FIG. 3 is a cross-sectional diagram of a humidification water tank provided in an embodiment of the present application, and FIG. 4 is an exploded schematic diagram of a humidifying water tank provided in an embodiment of the present application. A humidifying water tank 1 is applied to ventilation therapy equipment to humidify the airflow flowing into the water tank. The humidifying water tank 1 may be applied in the field of breathing machines, but is not limited thereto. The ventilation therapy equipment includes but is not limited to breathing assistance equipment such as a breathing machine and an oxygen therapy instrument.

The humidifying water tank 1 includes a shell 10, a base plate 20, a fan blade 30, a buoy 40, a fixed platen 50, and a sealing ring 60.

One end of the shell 10 is provided with an opening 101, and the other end of the shell 10 is provided with an air outlet 102, an air inlet 103, and a water injection port 104. In this embodiment, the entire shell 10 is in a hollow cylindrical shape, and the opening 101, the air outlet 102, the air inlet 103, and the water injection port 104 are arranged at two ends of the cylinder separately.

In this embodiment, the air inlet 103, the air outlet 102, and the water injection port 104 are all circular, the water injection port 104 is arranged in the center of the shell 10, and the air inlet 103 and the air outlet 102 are symmetrically arranged on two sides of the water injection port 104.

The shell 10 includes a fixed column 11, an air inlet pipe 12, an air inlet flow guide vane 13, an air outlet pipe 14, an air outlet flow guide vane 15, and a water injection pipe 17. The air inlet 103 is arranged at the air inlet pipe 12, the air outlet 102 is arranged at the air outlet pipe 14, and the water injection port 104 is arranged at the water injection pipe 17. In this embodiment, the water injection pipe 17 may be connected to an external water pipe. Specifically, the external water pipe may be fixedly connected to the water injection pipe 17 through an external connector; or the external water pipe may be formed by integrating a silicone hose and a connector, and directly connected to the water injection pipe 17, without any limitation here. One end of the external water pipe is connected to the water injection pipe 17, and the other end is connected to an external water bottle or an external water bag. The connector includes but is not limited to a Luer taper and the like.

The base plate 20 covers the opening 101 and encloses a receiving cavity 100 together with the shell 10. In this embodiment, the receiving cavity 100 is in communication with the air outlet 102, the air inlet 103, and the water injection port 104, respectively.

The receiving cavity 100 is filled with liquid injected from the water injection port 104. In this embodiment, the external water bottle or the external water bag is filled with liquid, and the liquid flows to the water injection pipe 17 through the external water pipe and flows into the receiving cavity 100 from the water injection port 104. That is, the liquid may be injected into the receiving cavity 100 from the water injection port. Preferably, the liquid is water.

The base plate 20 may be heated by an external heating component, so that the liquid is heated to form vapor. In this embodiment, the base plate 20 is made of a metal and may be used for heat conduction. The base plate 20 is in contact with the external heating component, and the heat generated by the external heating component may be conducted to the liquid through the base plate 20, thereby heating the liquid in the receiving cavity 100 to form vapor. Correspondingly, when the liquid is water, the vapor is water vapor.

The fixed column 11 extends from the periphery of the water injection port 104 towards the receiving cavity 100. In this embodiment, the fixed column 11 is provided with a limiting hole 110, and the limiting hole 110 is in communication with the water injection port 104 and the receiving cavity 100. Specifically, the fixed column 11 includes a plurality of column bodies 111, the column bodies 111 are arranged at intervals on the periphery of the water injection port 104, and the limiting hole 110 is formed between the column bodies 111. In this embodiment, a quantity of the column bodies 111 is four, and the four column bodies 111 are symmetrically arranged on the periphery of the water injection port 104. In some feasible embodiments, the quantity of the column bodies 111 may be configured according to an actual situation, and is not limited here.

Both the air inlet pipe 12 and the air outlet pipe 14 extend away from the receiving cavity 100 from a surface of the shell 10 to form hollow cylinders. In this embodiment, the air inlet flow guide vane 13 is connected to an end, close to the receiving cavity 100, of the air inlet pipe 12, and the air outlet flow guide vane 15 is connected to an end, close to the receiving cavity 100, of the air outlet pipe 14. An air inlet port 123 is formed between the air inlet flow guide vane 13 and the air inlet pipe 12, and the air inlet port 123 is in communication with the air inlet 103 and the receiving cavity 100. An air vent 145 is formed between the air outlet flow guide vane 15 and the air outlet pipe 14, and the air vent 145 is in communication with the air outlet 102 and the receiving cavity 100.

Specifically, a portion of the air inlet flow guide vane 13 is connected to the air inlet pipe 12, and the air inlet flow guide vane 13 obliquely connected to the air inlet pipe 12 at an angle with a bottom of the air inlet pipe 12; a portion of the air outlet flow guide vane 15 is connected to the air outlet pipe 14, and the air outlet flow deflector 15 is obliquely connected to the air outlet pipe 14 at an angle with a bottom of the air outlet pipe 14.

The fan blade 30 is accommodated in the receiving cavity 100 and rotatably mounted on the shell 10. In this embodiment, the fan blade 30 is arranged on the fixed column 11. The fan blade 30 includes a fixing portion 32 and a plurality of blades 31. The fixing portion 32 is provided with a fixing hole 320, and the fan blade 30 is sheathed on the fixed column 11 through the fixing hole 320. The blades 31 are arranged at intervals on a side, away from the fixed column 11, of the fixing portion 32. Each blade 31 includes a blade surface 310, and both the air inlet port 123 and the air vent 145 face toward the blade surface 310. Specifically, each blade 31 is connected to the fixing portion 32 at an angle, and both the air inlet port 123 and the air vent 145 perpendicularly face toward the blade surface 310 or at an angle close to 90°.

There is a gap between the fixing portion 32 and the fixed column 11. The fixed platen 50 is arranged on a side, away from the water injection port 104, of the fixing portion 32 and fixed with the fixed column 11. It should be understood that the fan blade 30 is fixed to the fixed column 11 through the fixed platen 50. Due to the gap between the fixing portion 32 and the fixed column 11, the fan blade 30 can rotate about the fixed column 11.

When airflow enters the receiving cavity 100 from the air inlet 103, the airflow can push the fan blade 30 to rotate about the fixed column 11 to form a wind force, and the airflow and vapor are accelerated and mixed under the rotation of the fan blade 30 to form a mixed gas. The mixed gas can flow out from the air outlet 102 under the action of the wind force generated by the fan blade 30. It should be understood that the airflow from the air inlet 103 can just act on the blade surfaces 310 through the air inlet port 123, so that the airflow can better push the fan blade 30 to rotate. The rotating fan blade 30 agitates the vapor in the receiving cavity 100, and the incoming airflow and vapor are fully mixed and then flow out from the air outlet 102, thereby improving a humidification effect. Meanwhile, the air inlet port 123 formed by the air inlet flow guide vane 13 and the air inlet pipe 12 enables the inflow direction of the airflow to be perpendicular or close to 90° to the blade surfaces 310, thereby better driving the rotation of the fan blade 30. The air vent 145 formed by the air outlet flow guide vane 15 and the air outlet pipe 14 enables the outflow direction of the mixed gas to be perpendicular or close to 90° to the blade surfaces 310, thereby better driving the mixed gas to flow out of the receiving cavity 100.

The air inlet pipe 12 is connected to breathing assistance equipment such as a pneumatic ventilator or an oxygen therapy instrument. The airflow generated by the breathing assistance equipment enters the air inlet pipe 12 from the air inlet 103, and acts on the blade surfaces 310 under the guidance of the air inlet flow guide vane 13. The fan blade 30 rotates under the action of the airflow and agitates the airflow and water vapor formed by heating and evaporation, and the airflow and the water vapor are fully mixed to form a mixed gas. The mixed gas flows out from the receiving cavity 100 through the air vent 145 and is inhaled by a user through a pipeline, a mask, and the like, which are externally connected, of the user thereby significantly improving the humidification rate of the airflow and the effect of breathing therapy.

In this embodiment, since the air inlet pipe 12, the air inlet flow guide vane 13, the air outlet pipe 14, and the air outlet flow guide vane 15 are arranged symmetrically, the air may flow in from the air inlet 103, and the mixed gas may flow out from the air outlet 102; or the air may flow in from the air outlet 102, and the mixed gas may flow out from the air inlet 103. That is, the air inlet 103 and the air outlet 102 have the same function. When a pipe externally connected to the air inlet pipe 12 introduces the airflow into the receiving cavity 100, the corresponding air inlet 103 of the air inlet pipe 12 plays a role in air intake. When a pipe externally connected to the air outlet pipe 14 introduces the airflow into the receiving cavity 100, the corresponding air outlet 102 of the air outlet pipe 14 plays a role in air intake. The difference in the air inlet pipe only affects the direction of rotation of the fan blade 30, but does not affect other functions of the humidifying water tank 1.

The buoy 40 is accommodated in the receiving cavity 100. One end of the buoy 40 extends into the limiting hole 110, and a top of the buoy 40 is opposite to the water injection port 104. In this embodiment, the buoy 40 may move along the fixed column 11 towards or away from the water injection port 104 under the action of liquid. That is, the buoy 40 may move along the fixed column 11 towards the water injection port 104, and the buoy 40 may also move along the fixed column 11 away from the water injection port 104.

The buoy 40 includes a main body 41, a protrusion 42, and columns 43. The protrusion 42 and the columns 43 are arranged on two sides of the main body 41 separately. In this embodiment, the protrusion 42 protrudes from one side of the main body 41 away from the main body 41. The interior of the main body 41 and the interior of the protrusion 42 are hollow, in communication, and filled with gas, so that the buoy 40 can rise or fall within the receiving cavity 100 under the buoyancy of liquid as the height of the liquid changes, which means that it move towards the water injection port 104 or away from the water injection port 104.

The protrusion 42 extends into the limiting hole 110, and an end, away from the main body 41, of the protrusion 42 is provided with a sealing element 44. The buoy 40 may move towards the water injection port 104 until the protrusion 42 abuts against the shell 10, and the buoy 40 may move away from the water injection port 104 until the columns 43 abut against the base plate 20. It should be understood that the buoy 40 is used for controlling the total amount of liquid injected into the receiving cavity 100. When the liquid injected into the receiving cavity 100 reaches a preset height, the buoy 40 moves towards the water injection port 104 under the buoyancy of the liquid until the protrusion 42 abuts against the shell 10. When the vapor in the receiving cavity 100 is carried out by the airflow and the height of the liquid drops, the buoy 40 falls accordingly and moves away from the water injection port 104 until the columns 43 abut against the base plate 20. In this embodiment, when the protrusion 42 abuts against the shell 10, the sealing element 44 blocks the water injection port 104 to stop the liquid from continuing to flow into the receiving cavity 100, so as to prevent excessive liquid injected into the receiving cavity 100 from affecting the humidification rate due to a long heating time. When the buoy 40 begins to move away from the water injection port 104 and the sealing element 44 does not block the water injection port 104, the water injection port 104 is in communication with the receiving cavity 100 again, and the liquid continues to be injected into the receiving cavity 100 to maintain the liquid at the preset height, and so on. It should be understood that, when the liquid is not injected into the receiving cavity 100 anymore, the buoy 40 can ultimately be fixed in the receiving cavity 100 through the abutment of the columns 43 against the base plate 20 with the emission of vapor. Specifically, the height of the buoy 40 is greater than the distance between the fixed column 11 and the base plate 20. The sealing element 44 is made of silicone.

The sealing ring 60 is clamped between the end, provided with the opening 101, of the shell 10 and the base plate 20. In this embodiment, the shell 10 further includes a limiting edge 16, and the limiting edge 16 is arranged at the end, provided with the opening 101, of the shell 10. Specifically, the limiting edge 16 extends from an edge of the shell 10 away from the receiving cavity 100. The base plate 20 includes a body 21 and a limiting edge 22. The limiting edge 22 extends from an edge of the body 21 towards the air inlet 103, and is bent and extended towards the center of the body 21. A limiting groove 200 is formed between the limiting edge 22 and the body 21.

In this embodiment, the limiting edge 16 is accommodated in the limiting groove 200, so that the base plate 20 covers the opening 101. The sealing ring 60 is accommodated in the limiting groove 200 and clamped between the limiting edge 16 and the body 21, and undergoes elastic deformation under the action of the limiting edge 16 and the body 21. The sealing ring 60 is made of silicone.

In the foregoing embodiment, through the cooperation of the air outlet, the air inlet, and the fan blade, the airflow flowing into the receiving cavity from the air inlet can drive the fan blade in the receiving cavity to rotate through the kinetic energy of the air, and the rotating fan blade can drive the airflow to the air outlet to flow out of the receiving cavity. The base plate can heat the liquid flowing in from the water injection port to generate vapor. The rotation of the fan blade can agitate the airflow and vapor in the receiving cavity, so that the air and the vapor are fully mixed to form a mixed gas with a high humidification rate. The mixed gas flows out from the air outlet under the rotation of the fan blade. The process of agitating the airflow and the vapor by the fan blade is beneficial to improving the humidification rate of the humidifying water tank.

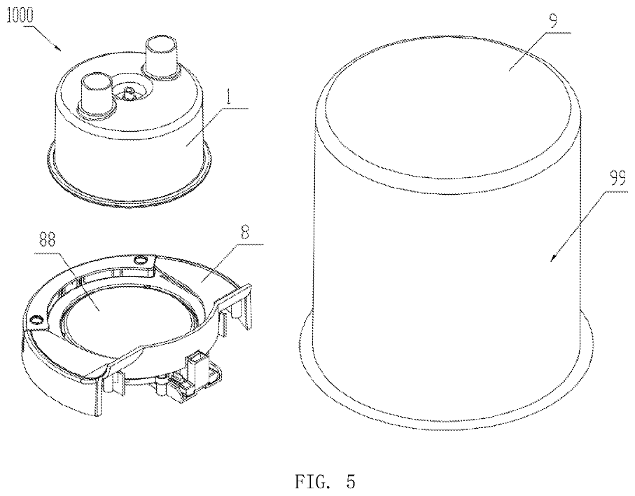

FIG. 5 is an exploded schematic diagram of ventilation therapy equipment provided in an embodiment of the present application. Ventilation therapy equipment 1000 includes but is not limited to breathing assistance equipment such as a breathing machine and an oxygen therapy instrument. The ventilation therapy equipment 1000 includes a basev b b 8, a housing 9, and a humidifying water tank 1. A specific structure of the humidifying water tank 1 refers to the foregoing embodiment.

In this embodiment, the housing 9 is provided with an accommodating cavity 99, and both the humidifying water tank 1 and the base 8 are accommodated in the accommodating cavity 99. Specifically, the humidifying water tank 1 is fixed to the base 8, and the base 8 includes a heating component 88. The heating component 88 is in contact with the base plate 20 to transfer the heat generated to the base plate 20.

The ventilation therapy equipment 1000 employs all the technical solutions of all the foregoing embodiments, and therefore has at least all the beneficial effects brought by the technical solutions of the foregoing embodiments, which will not be repeated here.

Apparently, those skilled in the art can make various modifications and variations to the present application without departing from the spirit and scope of the present application. Provided that these modifications and variations of the present application fall into the scope of the claims of the present application and equivalent technologies thereof, the present application is intended to include these modifications and variations.

What are listed above are merely preferred embodiments of the present application, and are certainly not intended to limit the scope of the claims of the present application. Therefore, equivalent variations made in accordance with the claims of the present application still fall within the scope of the present application.

1. A humidifying water tank, comprising:
a shell, one end of the shell provided with an opening, and the other end of the shell provided with an air outlet, and an air inlet;
a base plate, covering the opening and enclosing a receiving cavity together with the shell, the receiving cavity being in communication with the air outlet and the air inlet respectively; the receiving cavity being configured to receive liquid, the base plate being heated by an external heating component to enable the liquid being heated to form vapor;
a fan blade, being accommodated in the receiving cavity and rotatably mounted on the shell;
Wherein when airflow enters the receiving cavity from the air inlet, the airflow can push the fan blade to rotate about the fixed column to form the wind force, and the airflow and vapor are accelerated and mixed under the rotation of the fan blade to form a mixed gas; the mixed gas can flow out from the air outlet under an action of the wind force generated by the fan blade.

2. The humidifying water tank of claim 1, wherein the shell further comprises an air inlet pipe, and an air inlet flow guide vane, the air inlet is arranged at the air inlet pipe, the air inlet flow guide vane is connected to an end, close to the receiving cavity, of the air inlet pipe, an air inlet port is formed between the air inlet flow guide vane and the air inlet pipe, and the air inlet port is in communication with the air inlet and the receiving cavity, the fan blade includes a plurality of blades, each blade includes a blade surface, and the air inlet port faces toward the blade surface.

3. The humidifying water tank of claim 2, wherein the shell further defines a water injection port, the shell further comprises a fixed column, the fixed column extends from the periphery of the water injection port towards the receiving cavity, the fixed column is provided with a limiting hole, the limiting hole is in communication with the water injection port and the receiving cavity, the liquid may be injected into the receiving cavity from the water injection port, the humidifying water tank further comprises a buoy, accommodated in the receiving cavity, one end of the buoy extends into the limiting hole, and a top of the buoy is opposite to the water injection port, the buoy moves along the fixed column towards or away from the water injection port under the action of liquid.

4. The humidifying water tank of claim 3, wherein the buoy comprises a main body, a protrusion, and columns, the protrusion and the columns are arranged on two sides of the main body separately, the protrusion protrudes from one side of the main body away from the main body, the protrusion extends into the limiting hole, an end, away from the main body, of the protrusion is provided with a sealing element, the buoy moves away from the water injection port until the columns abut against the base plate, when the protrusion abuts against the shell, the sealing element blocks the water injection port.

5. The humidifying water tank of claim 3, wherein the fan blade further comprises a fixing portion, the fixing portion is provided with a fixing hole, and the fan blade is sheathed on the fixed column through the fixing hole, a gap is provided between the fixing portion and the fixed column, the blades are arranged at intervals on a side, away from the fixed column, the humidifying water tank further includes arranged on a side, away from the water injection port, and fixed with the fixed column.

6. The humidifying water tank of claim 3, wherein the shell further comprises air outlet pipe and air outlet flow deflector, the air outlet is arranged at the air outlet pipe, the air outlet flow guide vane is connected to an end, close to the receiving cavity, an air vent is formed between the air outlet flow guide vane and the air outlet pipe, the air vent is in communication with the air outlet and the receiving cavity, the air vent face toward the blade surface, the air inlet and the air outlet are symmetrically arranged on two sides of the water injection port.

7. The humidifying water tank of claim 1, wherein the humidifying water tank further comprises a sealing ring, the sealing ring is clamped between the end, provided with the opening, of the shell and the base plate, the base plate is made of a metal.

8. The humidifying water tank of claim 1, wherein the shell further includes a limiting edge, and the limiting edge is arranged at the end, provided with the opening, of the shell, the base plate includes a body and a limiting edge, the limiting edge extends from an edge of the body towards the air inlet, and is bent and extended towards the center of the body, a limiting groove is formed between the limiting edge and the body, the limiting edge is accommodated in the limiting groove, the sealing ring is clamped between the limiting edge and the body.

9. A ventilation therapy equipment, comprising:
a housing, and
a humidifying water tank being accommodated in the housing, the humidifying water tank comprising:
a shell, one end of the shell provided with an opening, and the other end of the shell provided with an air outlet, and an air inlet;
a base plate, covering the opening and enclosing a receiving cavity together with the shell, the receiving cavity being in communication with the air outlet and the air inlet respectively; the receiving cavity being configured to receive liquid, the base plate being heated by an external heating component to enable the liquid being heated to form vapor;
a fan blade, being accommodated in the receiving cavity and rotatably mounted on the shell;
When airflow enters the receiving cavity from the air inlet, the airflow can push the fan blade to rotate about the fixed column to form the wind force, and the airflow and vapor are accelerated and mixed under the rotation of the fan blade to form a mixed gas; the mixed gas can flow out from the air outlet under an action of the wind force generated by the fan blade.

10. The ventilation therapy equipment of claim 9, wherein the shell further comprises an air inlet pipe, and an air inlet flow guide vane, the air inlet is arranged at the air inlet pipe, the air inlet flow guide vane is connected to an end, close to the receiving cavity, of the air inlet pipe, an air inlet port is formed between the air inlet flow guide vane and the air inlet pipe, and the air inlet port is in communication with the air inlet and the receiving cavity, the fan blade includes a plurality of blades, each blade includes a blade surface, and the air inlet port faces toward the blade surface.

11. The ventilation therapy equipment of claim 10, wherein the shell further defines a water injection port, the shell further comprises a fixed column, the fixed column extends from the periphery of the water injection port towards the receiving cavity, the fixed column is provided with a limiting hole, the limiting hole is in communication with the water injection port and the receiving cavity, the liquid may be injected into the receiving cavity from the water injection port, the humidifying water tank further comprises a buoy, accommodated in the receiving cavity, one end of the buoy extends into the limiting hole, and a top of the buoy is opposite to the water injection port, the buoy moves along the fixed column towards or away from the water injection port under the action of liquid.

12. The ventilation therapy equipment of claim 11, wherein the buoy comprises a main body, a protrusion, and columns, the protrusion and the columns are arranged on two sides of the main body separately, the protrusion protrudes from one side of the main body away from the main body, the protrusion extends into the limiting hole, an end, away from the main body, of the protrusion is provided with a sealing element, the buoy moves away from the water injection port until the columns abut against the base plate, when the protrusion abuts against the shell, the sealing element blocks the water injection port.

13. The ventilation therapy equipment of claim 12, wherein the fan blade further comprises a fixing portion, the fixing portion is provided with a fixing hole, and the fan blade is sheathed on the fixed column through the fixing hole, a gap is provided between the fixing portion and the fixed column, the blades are arranged at intervals on a side, away from the fixed column, the humidifying water tank further includes arranged on a side, away from the water injection port, and fixed with the fixed column.

14. The ventilation therapy equipment of claim 12, wherein the shell further comprises air outlet pipe and air outlet flow deflector, the air outlet is arranged at the air outlet pipe, the air outlet flow guide vane is connected to an end, close to the receiving cavity, an air vent is formed between the air outlet flow guide vane and the air outlet pipe, the air vent is in communication with the air outlet and the receiving cavity, the air vent face toward the blade surface, the air inlet and the air outlet are symmetrically arranged on two sides of the water injection port.

15. The ventilation therapy equipment of claim 9, wherein the humidifying water tank further comprises a sealing ring, the sealing ring is clamped between the end, provided with the opening, of the shell and the base plate, the base plate is made of a metal.

16. The ventilation therapy equipment of claim 9, wherein the shell further includes a limiting edge, and the limiting edge is arranged at the end, provided with the opening, of the shell, the base plate includes a body and a limiting edge, the limiting edge extends from an edge of the body towards the air inlet, and is bent and extended towards the center of the body, a limiting groove is formed between the limiting edge and the body, the limiting edge is accommodated in the limiting groove, the sealing ring is clamped between the limiting edge and the body.

17. The ventilation therapy equipment of claim 9, wherein the ventilation therapy equipment further comprises a base receiving in the receiving cavity, the humidifying water tank is fixed to the base, the base includes a heating component.

18. A humidifying water tank, comprising:
a shell, the shell provided with an opening, an air outlet, and an air inlet;
a base plate, covering the opening and enclosing a receiving cavity together with the shell, the receiving cavity being in communication with the air outlet and the air inlet respectively; the receiving cavity being configured to receive liquid, the base plate being heated by an external heating component to enable the liquid being heated to form vapor; and
a fan blade, being accommodated and rotated in the receiving cavity, airflow being capable of entering the receiving cavity from the air inlet; during the rotation of the fan blade, the airflow being mixed with the vapor to form mixed gas, and the mixed gas being capable of flowing out from the air outlet.

19. The humidifying water tank of claim 18, wherein the shell further comprises an air inlet pipe, and an air inlet flow guide vane, the air inlet is arranged at the air inlet pipe, the air inlet flow guide vane is connected to an end, close to the receiving cavity, of the air inlet pipe, an air inlet port is formed between the air inlet flow guide vane and the air inlet pipe, and the air inlet port is in communication with the air inlet and the receiving cavity, the fan blade includes a plurality of blades, each blade includes a blade surface, and the air inlet port faces toward the blade surface.

20. The humidifying water tank of claim 18, wherein the shell further defines a water injection port, the shell further comprises a fixed column, the fixed column extends from the periphery of the water injection port towards the receiving cavity, the fixed column is provided with a limiting hole, the limiting hole is in communication with the water injection port and the receiving cavity, the liquid may be injected into the receiving cavity from the water injection port, the humidifying water tank further comprises a buoy, accommodated in the receiving cavity, one end of the buoy extends into the limiting hole, and a top of the buoy is opposite to the water injection port, the buoy moves along the fixed column towards or away from the water injection port under the action of liquid.
